(19) United States
(12) Patent Application Publication
(10) Pub. No.: US 2015/0119692 A1
(43) Pub. Date: Apr. 30, 2015

(54) NON-INVASIVE EVALUATION OF FIT AND SIZING OF DEVICE PRIOR TO IMPLANT

(71) Applicant: Medtronic, Inc., Minneapolis, MN (US)

(72) Inventors: Brian McHenry, Minneapolis, MN (US); Victor Okali, Brooklyn Center, MN (US); Alexander Hill, Blaine, MN (US)

(21) Appl. No.: 14/519,891

(22) Filed: Oct. 21, 2014

Related U.S. Application Data
(60) Provisional application No. 61/895,495, filed on Oct. 25, 2013.

(51) Int. Cl. A61B 5/00; A61B 5/055; A61B 6/00; A61B 6/03; A61B 19/00; A61B 8/08

(52) U.S. Cl. CPC A61B 5/4851; A61B 19/50; A61B 8/0883; A61B 6/503; A61B 6/032; A61B 5/055; A61B 2019/507
USPC 600/411; 600/439; 600/436; 600/427

(57) ABSTRACT

Maximum and minimum dimensional characteristics of a conduit are determined by imaging a portion of the conduit into which an expandable medical device is to be implanted at appropriate points in the cardiac cycle (or other suitable cycle, such as the respiratory cycle, or at other suitable times). The dimensional characteristics of the conduits within the portion of interest may be evaluated and compared to the maximum and minimum dimensional characteristics of the medical device to be implanted in the conduit to determine whether the medical device is of an appropriate size for properly fitting in the conduit.

### RELATED APPLICATION

This application claims the benefit of U.S. Provisional Patent Application No. 61/895,495, filed on Oct. 25, 2014, which provisional patent application is hereby incorporated herein by reference in its entirety to the extent that it does not conflict with the disclosure presented herein.

### FIELD

This disclosure generally relates to, among other things, methods, devices, systems for non-invasive evaluation of fit and sizing of medical device prior to implantation; particularly transcatheter medical devices such as prosthetic heart valves, stents, grafts and the like.

### BACKGROUND

A number of implantable medical devices are available for replacement or repair of a conduit, vessel, or organ structure within a patient. Such devices include homografts, xenografts, bioprostheses such as replacement valves, stents, and the like. Many of such devices are implantable via transcatheter procedures, which are procedures where a catheter, in which or about which a transcatheter medical device is disposed, is advanced within a vessel, organ structure or other conduit to a desired location where the implantable medical device is deployed. The device may be self-expandable, balloon-expandable, or the like. However, it may not be suitably known whether the device will appropriately fit and function within the vessel or organ structure until after the device is placed within the vessel or organ structure.

### SUMMARY

In various embodiments, this disclosure describes, among other things, methods, devices, and systems for non-invasive evaluation of fit and sizing of medical device prior to implantation. The implantable devices may be expandable from a collapsed or constrained configuration to an expanded configuration and may interact with the interior wall of a vessel, organ structure, or other bioprosthetic or natural conduit, or the like via interference fit when expanded. Examples of expandable implantable medical devices include transcatheter medical devices such as prosthetic heart valves, stents, grafts and the like.

In various embodiments described herein, one or more characteristics or dimensions of a vessel, organ structure or other bioprosthetic or natural conduit is assessed, measured or determined. As used hereinafter, “conduit” will be used to collectively refer to a bioprosthetic or natural conduit, vessel, or organ structure into which an expandable medical device may be implanted. In various embodiments described herein, one or more maximum and minimum characteristics or dimensions such as diameters, perimeters, lengths, areas, including cross-sectional area or surface area, etc., of a conduit are determined by imaging a portion of the conduit into which the device is to be implanted at appropriate points in the cardiac cycle (or other appropriate cycle, such as the respiratory cycle, etc.). As used hereinafter, “dimensional characteristic” will be used to refer collectively to perimeter, diameter (including perimeter derived diameter, area derived diameter, average diameter, major diameter, minor diameter, etc.), area (such as cross-sectional area, surface area, etc.), length, aspect ratio, shape and the like. For example, the dimensional characteristics of conduits within the portion of interest may be evaluated and compared to the maximum, expanded and minimum, contracted dimensional characteristics of the expandable medical device to be implanted in the conduit. If the dimensional characteristics of the conduit are within desired ranges of dimensional characteristics for proper interference fit of the expanded medical device, one may have improved confidence that the expandable medical device will properly fit and function when implanted.

As used herein, a “minimum, contracted” dimensional characteristic of an implantable medical device is a dimensional characteristic in which the device is constrained, such as by a sheath or temperature (e.g., for crimped devices that self-expand upon temperature change) or the like, but does not necessarily mean that the device is not capable of achieving smaller dimensional characteristics. Typically, the minimum, contracted dimensional characteristics of an implantable medical device are dimensional characteristics of the device during an implant procedure prior to deployment or expansion of the device. As used herein, a “maximum, expanded” dimensional characteristic of an expandable implantable medical device is a maximum dimensional characteristic of the device in a fully expanded configuration absent external forces. It will be understood that, when implanted, an expandable medical device may be biased towards the maximum, expanded configuration but may not achieve the full maximum, expanded state due to forces of the conduit or surrounding tissue, etc. on the device.

In various embodiments described herein, methods are disclosed for determining whether an expandable medical device is appropriate for implanting in a conduit of a patient based on one or more dimensional characteristics of the conduit as compared to dimensional characteristics of the expandable medical device. In various embodiments described herein, methods are disclosed for selecting an expandable medical device (among a plurality of devices) for implanting in a conduit of a patient based on one or more dimensional characteristics of conduit as compared to one or more dimensional characteristics of the expandable medical devices. In various embodiments described herein, methods are disclosed for identifying an appropriate region of a conduit for implanting an expandable medical device based on one or more dimensional characteristics of the conduit as compared to one or more dimensional characteristics of the expandable medical device. In various embodiments described herein, methods are disclosed for determining an appropriate dimensional characteristic specifications for an expandable medical device for implanting in a desired portion of a conduit of a patient based on dimensional characteristics of the conduit (either of a given patient or a population of subjects). The medical device may then be manufactured to the appropriate specifications.

In some embodiments, a non-invasive method for determining whether an expandable medical device is appropriate for implanting in a conduit of patient includes imaging a portion of the conduit of the patient into which the medical device is to be implanted, wherein the imaging is performed during (i) a portion of the cardiac cycle (or other suitable cycle, such as respiratory cycle, etc., or other suitable time) at which one or more dimensional characteristics of the conduit are at or near minimums and (ii) a portion of the cardiac cycle (or other suitable cycle, such as respiratory cycle, etc., or other suitable time) at which one or more dimension characteristics of the conduit are at or near maximums (generally to obtain information regarding extremes of the dimensional characteristics). The method further includes deriving, from the imaging, minimum and maximum dimensional characteristics (roughly the extremes of dimensional characteristics) such as perimeter of the conduit at a plurality of locations along a length of the portion of the conduit. The method also includes determining that the medical device is appropriate for implantation if the maximum and minimum dimensional characteristics of the conduit at the predetermined locations along the length of the conduit are within predetermined ranges of maximum, expanded and minimum, contracted dimensional characteristics of the medical device at predetermined locations along the length of the medical device.

In some embodiments, a non-invasive method for selecting an expandable medical device for implanting in a conduit of a patient includes imaging a portion of the conduit of the patient into which the medical device is to be implanted, wherein the imaging is performed during at a plurality of portions of the cardiac cycle, such as (i) a portion of the cardiac cycle (or other suitable cycle, such as respiratory cycle, etc., or other suitable time) at which the dimensional characteristics of the conduit are at or near minimums and (ii) a portion of the cardiac cycle (or other suitable cycle, such as respiratory cycle, etc., or other suitable time) at which the dimensional characteristics of the conduit are at or near maximums. The method further includes deriving, from the imaging, minimum and maximum dimensional characteristics of the conduit at a plurality of locations along a length of the portion of the conduit. The method also includes selecting a medical device as appropriate for implanting in the patient if the maximum and minimum dimensional characteristics of the conduit at the predetermined locations along the length of the conduit are within predetermined ranges of maximum, expanded and minimum, contracted dimensional characteristics of the medical at predetermined locations along the length of the medical device.

In some embodiments, a non-invasive method for selecting an appropriate region of a conduit of a patient for implanting an expandable medical device includes imaging a portion of the conduit of the patient into which the medical device is to be implanted, wherein the imaging is performed during a plurality of portions of the cardiac cycle, such as (i) a portion of the cardiac cycle (or other suitable cycle, such as respiratory cycle, etc., or other suitable time) at which the dimensional characteristics of the conduit are at or near minimums and (ii) a portion of the cardiac cycle (or other suitable cycle, such as respiratory cycle, etc., or other suitable time) at which the dimensional characteristics of the conduit are at or near maximums. The method further includes deriving, from the imaging, minimum and maximum perimeter dimensions of the conduit at a plurality of locations along a length of the portion of the conduit. The method also includes determining whether a region in the portion of the conduit has maximum and minimum dimensional characteristics at predetermined distances along the length of the conduit in the region that are within predetermined ranges of maximum, expanded and minimum, contracted dimensional characteristics of the medical device at predetermined locations along the length of the medical device.

In some embodiments, a method for determining appropriate dimensional characteristic specifications for an expandable medical device for implanting in a conduit of a patient includes imaging a portion of the conduit of the patient into which the medical device is to be implanted, wherein the imaging is performed during a plurality of portions of the cardiac cycle, such as (i) a portion of the cardiac cycle (or other suitable cycle, such as respiratory cycle, etc., or other suitable time) at which the dimensional characteristics of the conduit are at or near minimums and (ii) a portion of the cardiac cycle (or other suitable cycle, such as respiratory cycle, etc., or other suitable time) at which the dimensional characteristics of the conduit are at or near maximums. The method further includes deriving, from the imaging, minimum and maximum dimensional characteristics of the conduit at a plurality of locations along a length of the portion of the conduit. The method also includes determining the appropriate dimensional characteristics at various locations along the length of the medical device as dimensional characteristics that are at predetermined preferred maximum and minimum dimensional characteristics at the plurality of locations along the length of the conduit.

The methods described herein may be computer-implemented methods. Non-transitory computer-readable media that, when implemented, cause a device to carry out the one or more methods are described herein. Devices employing such non-transitory computer-readable media or otherwise configured to carry out the methods are also described.

One or more embodiments of the devices, systems and methods described herein have one or more advantages relative to prior devices, systems and methods for evaluating fit and sizing of medical devices prior to implantation. Those of skill in the art, upon reading the present disclosure and accompanying drawings, will readily appreciate these advantages.

### BRIEF DESCRIPTION OF THE DRAWINGS

FIG. 1A is a schematic side view of an embodiment of a stented prosthetic heart valve in a natural, expanded arrangement.

FIG. 1B is a schematic side view of an embodiment of the prosthesis of FIG. 1A in a compressed or collapsed arrangement.

FIGS. 2A and 2B are schematic drawings illustrating the use of an embodiment of a delivery system for transcatheter delivery of an embodiment of a prosthetic heart valve to an aortic valve implantation site via an aortic arch of a patient.

FIG. 3 is schematic drawing of an embodiment of a three-dimensional right-heart model from multiphase CT, perimeter and length measurements.

FIG. 4 is a schematic drawing of an embodiment of a virtual device for implanting in a pulmonary artery overlaid with an embodiment of a model of right heart model showing the pulmonary artery.

FIG. 5 is a plot showing dimensions of an embodiment of a prosthetic heart valve along the length of the prosthetic valve, dimensions of a portion of a conduit along a length of a portion of the conduit into which the device may be implanted, and a desired region in which the perimeters of the conduit preferably fall over the entire cardiac cycle for the given heart valve.

FIGS. 6-7 are plots similar to the plot of FIG. 5.

FIG. 8 is a schematic drawing of an embodiment of a system 200 that may be used to execute embodiments of methods or processes described herein.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description several specific embodiments of devices, systems and methods are disclosed. It is to be understood that other embodiments are contemplated and may be made without departing from the scope or spirit of the present disclosure. The following detailed description, therefore, is not to be taken in a limiting sense.

This disclosure relates generally to, among other things, methods, devices, systems for non-invasive evaluation of fit and sizing of medical device prior to implantation. The implantable devices may be expandable from a collapsed configuration to an expanded configuration and may interact with an interior wall of a conduit via interference fit when expanded. Examples of expandable implantable medical devices that are expandable include transcatheter medical devices such as prosthetic heart valves, stents, grafts and the like.

While the focus of much of this disclosure is on prosthetic heart valves, it will be understood that the disclosure is applicable to other implantable medical devices including expandable or transcatheter implantable medical devices. As used herein, a “transcatheter” medical device is a device that is deliverable for implantation via a catheter, particularly a catheter that is capable of being advanced through a conduit of a patient. When used to deliver expandable medical devices, the transcatheter delivery systems typically includes a sheath or other suitable feature to retain the implantable device in a minimum, contracted configuration until the device is deployed at a desired implant site within a conduit. In some embodiments, the conduit of the patient is a conduit of the patient's circulatory system such as a heart, vein or artery. Of course, the conduit may be a conduit of the patient's respiratory system, excretory system, lymphatic system, digestive system, reproductive system, etc.

An “expandable” medical device is a device configured to have a collapsed configuration prior to or during implantation and to have an expanded configuration during or following implantation. Expandable devices typically have a maximum expanded configuration in a free state. When implanted and expanded, such devices tend to be biased towards their maximum expanded free state, but may not be “fully” expanded due to countervailing forces based, at least in part, on the patient's anatomy. Expandable medical devices include (i) self-expanding devices such as those formed, at least in part, by shape-memory materials, (ii) balloon-expandable devices, and (iii) devices that are expandable in any other suitable manner.

The length of an expandable medical device and collapsed and maximal, expanded dimensional characteristics along the length of the device (or at predetermined locations along the length of the device) may be compared to anatomy of a portion of a conduit into which the device is to be implanted to determine whether the device is of appropriate size or shape to properly fit within the portion of the conduit.

The anatomy of a conduit, such as a cardiac structure, may be determined by using any suitable imaging technique, such as magnetic resonance imaging (MRI), ultrasound imaging, computed tomography (CT) imaging, x-ray imaging (e.g., x-ray angiography), and the like. In various embodiments, imaging of a conduit occurs during (i) a portion of the cardiac cycle (or other suitable cycle, such as respiratory cycle, etc., or other suitable time) at which dimensional characteristics of the conduit (or a portion thereof) are at or near minimums and (ii) a portion of the cardiac cycle (or other suitable cycle, such as respiratory cycle, etc., or other suitable time) at which the dimensional characteristics of the conduit (or a portion thereof) are at or near maximums. For certain conduits or portions thereof, such as regions of the pulmonary artery, it has been determined that images captured near 30% phase of the cardiac cycle (at or near late systole) and images captured near 90% phase of the cardiac cycle (near end diastole) are effective at identifying maximum (or near maximum) and minimum (or near minimum) dimensional characteristics of the conduit. Of course, for other conduits or other regions of the same conduits, images captured at different phases of the cardiac cycle (or other suitable cycle, such as respiratory cycle, etc., or other suitable time) may provide images at or near minimum and maximum dimensional characteristics.

Minimum and maximum dimensional characteristics along the length of a portion of a conduit (or at predetermined locations along the length of the conduit) may be derived from imaging and may be compared to the length and minimal, contracted dimensional characteristics and maximal (i.e., fully) expanded dimensional characteristics along the length of the device to be implanted (or at predetermined locations along the length of the device). Data regarding the minimum dimensional characteristics along the length of the conduit may be used to assist in determining whether the device (and associated catheter, if employed) may be advanced through the conduit to the implant location and whether the device may maintain an expanded configuration (or partially expanded configuration in which the device will remain biased towards its expanded configuration) at the minimum conduit dimensional characteristics. Data regarding the maximum dimensional characteristics along the length of the portion conduit may be used to assist in determining whether the device may remain retained in the conduit. Preferably, the maximum dimensional characteristics of at least one location of the conduit in which the device is to be implanted is less than the maximum dimensional characteristics of the device at a corresponding location so that the device will remain anchored in the conduit via interference fit throughout the cardiac cycle (or other suitable cycle, such as respiratory cycle, etc., or other suitable time).

Accordingly, the minimum and maximum dimensional characteristics along the length of the portion of the conduit (or at predetermined locations along the length of the conduit) are preferably within predetermined ranges of minimum and maximum dimensional characteristics along corresponding locations of the device. For example, the conduit preferably has minimum dimensional characteristics that are sufficiently large to prevent collapse of the device (i.e., the device remains biased towards its expanded configuration), to ensure that the device does not prematurely wear, to ensure proper operation the device or the conduit in which the device is implanted, or the like. The conduit also preferably has maximum dimensional characteristics that are sufficiently small to ensure that the device remains in place via interference fit at the maximum conduit dimensional characteristics. The desired maximum and minimum conduit dimensional characteristics ranges and preferred maximum and minimum conduit dimensional characteristics will vary from device-to-device.

By way of example, for prosthetic heart valves, the maximum dimensional characteristics of the conduit at a location at which a distal portion of the prosthetic heart valve (valve outflow portion) is to be implanted is preferably from about 60% to about 95% of the maximum, expanded dimensional characteristics of the distal portion of the heart valve (preferably at a location of the distal portion of the device having the largest outer dimensional characteristics). For example, the maximum dimensional characteristics of the conduit at the location at which the distal portion of the prosthesis is to be implanted may be 85% or less than the maximum, expanded dimensional characteristics of the distal portion of the prosthetic heart valve.

By way of further example, for prosthetic heart valves, the maximum dimensional characteristics of the conduit at a location at which a proximal portion of the prosthetic heart valve (valve inflow portion) is to be implanted is preferably from about 60% to about 90% of the maximum, expanded dimensional characteristics of the proximal portion of the heart valve (preferably at a location of the proximal portion of the device having the largest outer dimensional characteristics). For example, the maximum dimensional characteristics of the conduit at the location at which the proximal portion of the prosthesis is to be implanted may be 85% or less, or 80% or less, than the maximum, expanded dimensional characteristics of the distal portion of the prosthetic heart valve.

The preferred ranges of maximum and minimum dimensional characteristics along the length of the conduit (for a given medical device) may be weighted at various locations in determining whether the conduit portion is suitable for implanting the device, determining which device (among a plurality of devices) is best suited for implant in the portion the conduit, or determining which conduit region (among a plurality regions) is best suited for implanting a particular device. For example, more weight may be given to a desired fit at a proximal region of the device than at a distal region of the device or a mid-region of the device. By way of another example, more weight may be given to a desired fit at a distal region of the device than at a proximal region of the device or a mid-region of the device. By way of yet another example, more weight may be given to a desired fit at both a proximal region of the device and at a distal region of the device than at a mid-region of the device. Of course another other suitable or desired weighting scheme may be employed to assist in determining whether the conduit portion is suitable for implanting the device, determining which device is best suited for implant in the portion the conduit, or determining which conduit region is best suited for implanting a device.

The methods, devices and systems described herein may be employed with any suitable implantable medical device, such as homografts, xenografts, stents and prosthetic valves. The implantable medical device may be an expandable medical device.

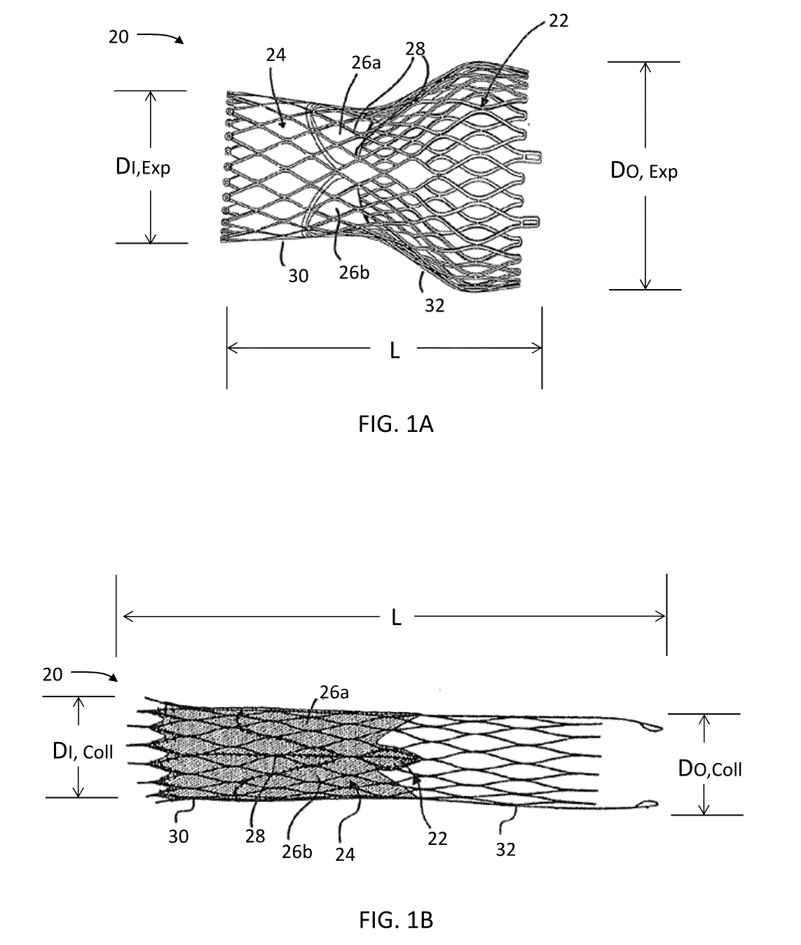

By way of example, one non-limiting example of a prosthetic heart valve 20 useful with devices and methods of the present disclosure is illustrated in FIG. 1A. As a point of reference, the prosthetic heart valve 20 is shown in a natural or expanded arrangement in the view of FIG. 1A. FIG. 1B illustrates the prosthetic heart valve 20 in a compressed arrangement (e.g., when compressively retained within an outer tube or sheath). The prosthetic heart valve 20 includes a stent or stent frame 22 and a valve structure 24. The stent frame 22 can assume any of the forms described above, and is generally constructed so as to be self- or otherwise-expandable from the compressed arrangement (FIG. 1B) to the natural, expanded arrangement (FIG. 1A). The valve structure 24 is assembled to the stent frame 22 and forms or provides two or more (typically three) leaflets 26a, 26b. The valve structure 24 can assume any of the forms described above, and can be assembled to the stent frame 22 in various manners, such as by sewing the valve structure 24 to one or more of the wire segments 28 defined by the stent frame 22.

One acceptable construction of the prosthetic heart valve 20 depicted in FIGS. 1A and 1B can be used for repairing a pulmonic valve. Of course, other shapes and sizes are envisioned to adapt to the specific anatomy of the valve to be repaired (e.g., stented prosthetic heart valves in accordance with the present disclosure can be shaped and/or sized for replacing a native mitral, aortic, or tricuspid valve). Regardless of the use, the depicted stent frame 22 defines an axial length L of the prosthetic heart valve 20. In the depicted embodiment, the valve structure 24 extends less than the entire length L of the stent frame 22. The valve structure 24 can be assembled to, and extend along, an inflow region 30 of the prosthetic heart valve 20, whereas an outflow region 32 is free of the valve structure 24 material. As a point of reference, “inflow” and “outflow” terminology is in reference to an arrangement of the prosthetic heart valve 20 upon final implantation relative to the native pulmonic valve (or other valve) being repaired. A wide variety of constructions are also acceptable and within the scope of the present disclosure. For example, in other embodiments, the valve structure 24 can extend along an entirety, or a near entirety, of a length of the stent frame 22.

As depicted, the outflow region 32 has an maximum expanded free-state diameter D_{O,Exp} and the inflow region 30 has an maximum expanded free-state diameter D_{I,Exp} (FIG. 1A), and the outflow region 32 has a collapsed diameter D_{O,Coll} and the inflow region 30 has a collapsed diameter D_{I,Coll} (FIG. 1B). As shown in, for example, FIG. 1A the diameter of the prosthetic heart valve 20 or stent frame 22 can vary along the length Lp of the valve or frame. The diameter at various locations along the length of the frame 22 or other suitable dimensions can be compared to appropriate dimensions of a conduit into which the prosthetic valve 20 prior to implanting the valve to non-invasively determine if the prosthetic valve will fit and function appropriately within the conduit once implanted.

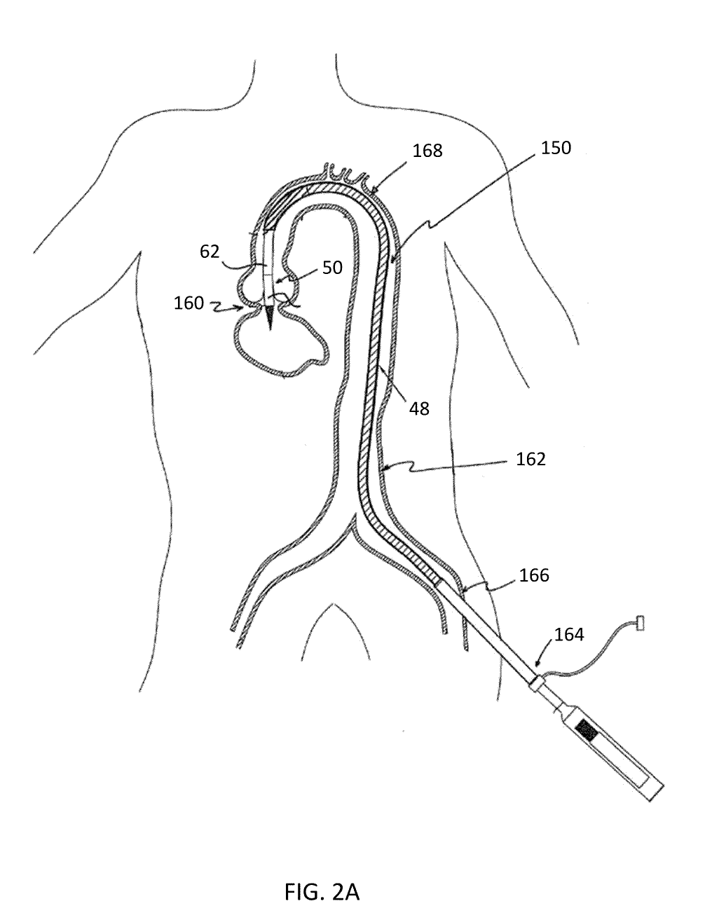

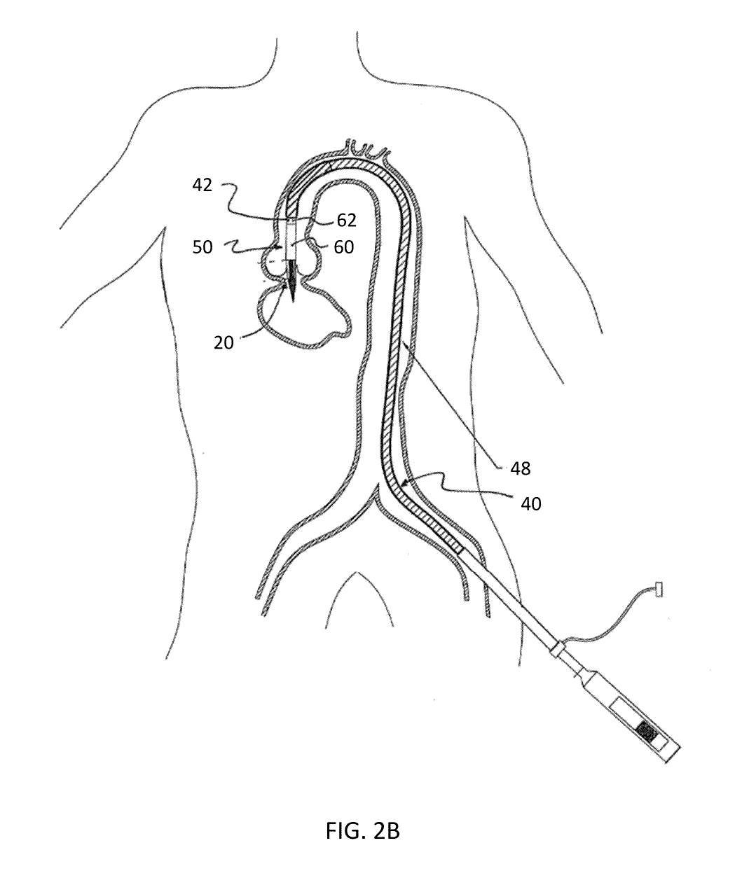

A prosthetic heart valve, such as depicted in FIGS. 1A-B, or other implantable medical device such as a graft or stent, may be implanted via a transcatheter procedure. One such procedure is schematically reflected in FIG. 2A in which device 150 is employed to repair a defective aortic valve 160. As shown, the device 150 (in the loaded state having a loaded prosthetic valve) is introduced into the patient's vasculature 162 (referenced generally) via an introducer device 164. The introducer device 164 provides a port or access to a femoral artery 166. From the femoral artery 166, capsule 50 (that otherwise compressively retains the prosthetic heart valve) is advanced via a retrograde approach through an aortic arch 168 (e.g., via iliac arteries). FIG. 2A depicts an optional outer stability tube 48 extending along a substantial length of delivery sheath assembly 42, with the distal end 130 being fairly proximate the capsule 50. FIG. 2B schematically illustrates deployment of the prosthetic heart valve 20 from the delivery system 40 via proximal retraction of the delivery sheath assembly 42, and in particular the capsule 50. A comparison of FIGS. 2A and 2B reveals that in the deployed state of FIG. 2B, the proximal segment 62 of the capsule 50 is retracted within the optional outer stability tube 48.

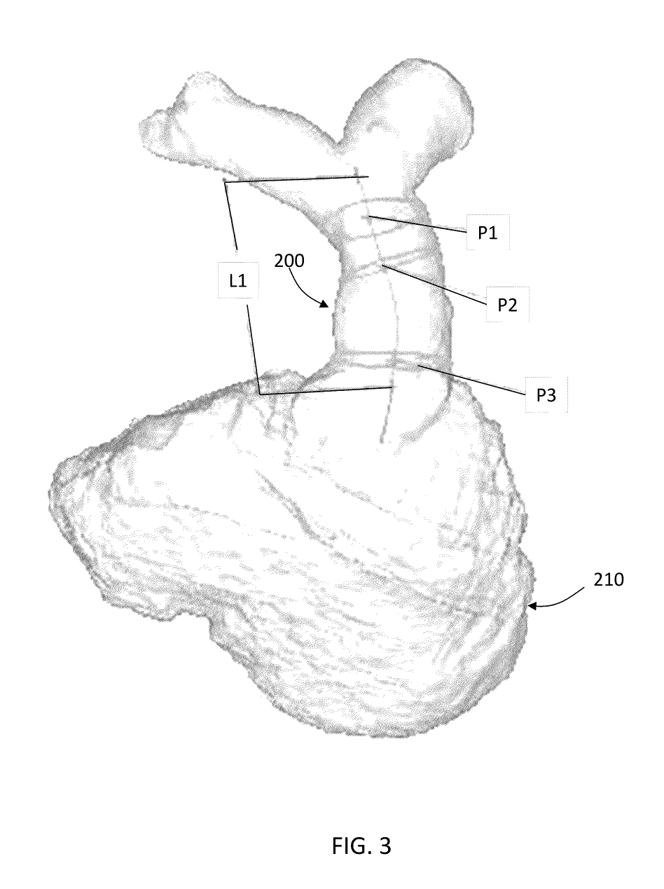

FIG. 3 depicts a three-dimensional right-heart model showing a portion of the right ventricle 210 and pulmonary artery 200. The model was obtained from multiphase computer tomography, perimeter and length measurements. The model may be useful for purposes of determining fit of, among other things, prosthetic pulmonary artery valves. In the model depicted, the length, L1, of the pulmonary artery 200 in which a prosthetic valve may be placed, as well as various diameters or perimeters (P1, P2, P3) at various locations along the length of the artery, are shown.

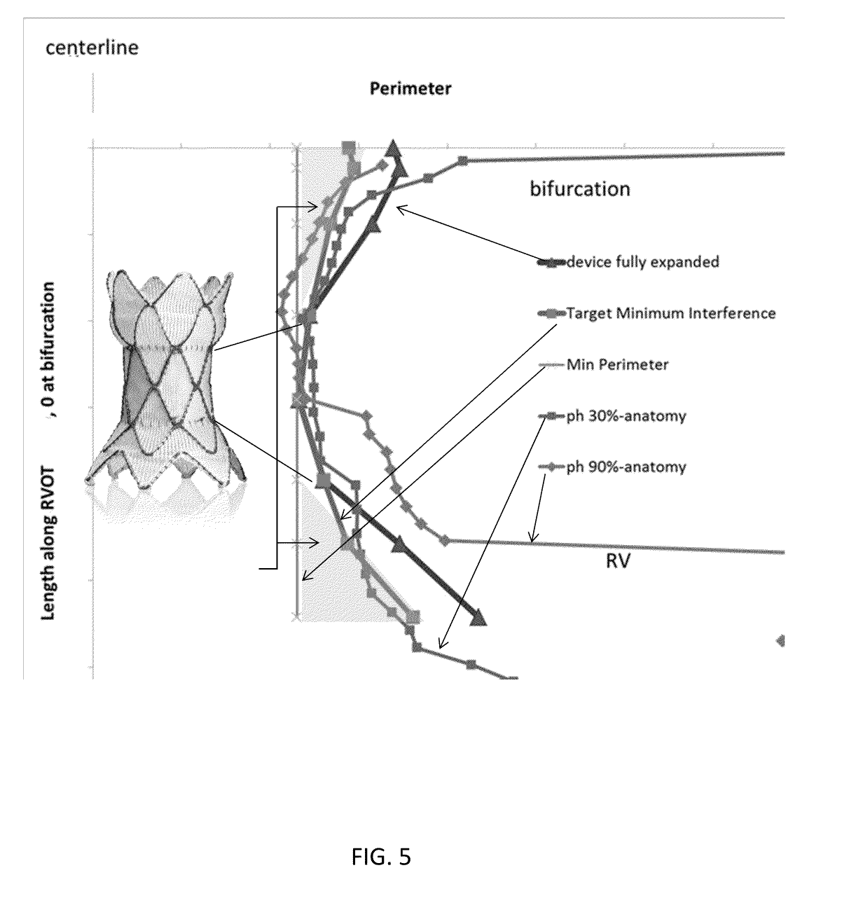

FIG. 3 is shown for purposes of illustration as an example of a model that may be useful for the devices and methods described herein. It will be understood that other suitable models of similar or different conduits, which may depend on the device to be implanted, may be employed. The model depicted in FIG. 3 is a three-dimensional visually displayed model, but may be a mathematical model, such as an explicit mathematic model, a plot (e.g. as depicted in FIG. 5) or any other suitable model for mapping, storing, accessing, etc. dimensional characteristics of a conduit. As indicated above, a combination of imaging and processing techniques may be employed to develop a model to determine appropriateness and acceptability of fit of a device in the conduit. In some embodiments, one or more of magnetic resonance imaging, ultrasound imaging, computed tomography imaging, and x-ray imaging are employed to develop a suitable model.

In many embodiments, a model includes minimum and maximum dimensional characteristics, such as diametric dimensions or perimeters and lengths, of the conduit into which the device is to be implanted. Because the dimensional characteristics of the conduit can change under certain physiological conditions, it can be useful account for such changes in the model of the conduit to determine suitableness of fit of the device to be implanted in the conduit. By way of example, the dimensional characteristics of conduits of the cardiovascular system, or portions thereof, change during the cardiac cycle. The cardiac cycle is a period from the beginning of one heart beat (0%) to the beginning of the next heart beat (100%). The inventors have found that the pulmonary artery experiences maximum diametric dimensions or perimeter at about 30% phase of the cardiac cycle (at or near late systole) and experiences minimum diametric dimensions or perimeter at about 90% phase of the cardiac cycle (near end diastole). Accordingly, in some embodiments where models of conduits of the cardiovascular system are developed, particularly models of the pulmonary artery, imaging of the conduit includes imaging within a range from about 25% to about 35% of the cardiac cycle and within a range from about 85% to about 95% of the cardiac cycle. For example, imaging of the conduit includes imaging within a range from about 27% to about 33% or at about 30% of the cardiac cycle and within a range from about 87% to about 93% or at about 90% of the cardiac cycle.

Because imaging of a conduit throughout the cardiac cycle for a sufficient number of cycles to obtain a reliable model of the conduit can be costly or impracticable, it can be beneficial to obtain images during limited periods of time or at one or more given points in time. For example, imaging of the conduit may be limited to imaging within or throughout a range from about 25% to about 35% of the cardiac cycle and within or throughout a range from about 85% to about 95% of the cardiac cycle, rather than continuously throughout the entire cardiac cycle. In some embodiments, imaging occurs at a point in time within a range from about 25% to about 35% of the cardiac cycle and at a point in time within a range from about 85% to about 95% of the cardiac cycle. For example, imaging of the conduit can occur at a point in time within a range from about 27% to about 33% or at about 30% of the cardiac cycle and at a point in time within a range from about 87% to about 93% or at about 90% of the cardiac cycle.

Obtained imaging data can be processed to develop minimum and maximum dimensional characteristics of a conduit in any suitable manner. For example, imaging may occur continuously over time to determine maximum and minimum parameters, may occur continuously over limited times around which the conduit is known or thought to be at minimum or maximum parameters, or may occur at one or more points in time within a range of times around which the conduit is known or thought to be at minimum or maximum parameters. Regardless of how the imaging data is obtained, a model (e.g., visual, mathematical, etc.) can preferably be formed based on imaging data to aid in determining whether an implantable device can be advanced through the modeled conduit in a collapsed or constrained state when the conduit, or portions thereof, are at minimum diametric or perimeter dimensions or can be retained within the conduit, e.g., through interference fit when the conduit, or portions thereof, are at maximum diametric or perimeter dimensions without collapse of the device at the minimum dimensional characteristics of the conduit. In some circumstances, such as where it is known or believed that the minimum dimensional characteristics of the conduit will permit advancement of a collapsed or constrained device and will be sufficiently large to avoid collapse of the device within the conduit, omission of minimal dimensional characteristics from the model may be suitable for purposes of expediency. Of course, minimal dimensional characteristics of the conduit may still have value in such circumstances.

In some embodiments, a physical model of a conduit into which a medical device is to be implanted can be made. The physical model may be used to verify fit or fit and function of the device in the conduit. Examples of physical models include stereolithographic models, 3D-printed models, and the like. Physical models may be made of one or both of maximum and minimum dimensional characteristics or dimensional characteristics based on imaging data obtained under different conditions (e.g., at different phases of the cardiac or other suitable cycle).

A model of a conduit as described herein may be based on imaging data from a specific patient or a population of patients. In some embodiments, a model is developed based on an imaging data from an individual patient, and the model is used to select or custom manufacture a device configured to suitably fit the conduit of that patient. In some embodiments, a model is based on imaging data obtained from a population of patients suffering from the same or a similar disorder. Such models may be used to develop devices that can be tailored to common anatomical features of the conduit in such patients.

In various embodiments, the methods, devices and systems are employed to determine fit, shape or sizing of a prosthetic heart valve for treatment of a right ventricular outflow tract (RVOT) anomaly. One such anomaly is Tetralogy of Fallot. Patients diagnosed as having Tetralogy of Fallot may be divided into two groups: (i) those with pulmonary stenosis (which accounts for about 85% of patients having RVOT anomalies and for which conduit placement was not considered), and (ii) those with pulmonary sensosis/Atresia for which repair of right ventricle to pulmonary artery conduit may be appropriate. Other RVOT anomaly patients for which repair of right ventricle to pulmonary artery conduit may be appropriate includes those diagnosed as having Truncus Arteriosus, Tranposition of Great Arteries, and Others.

In some embodiments, imaging data can be used to develop a transcatheter pulmonary valve intended for patients with pulmonary regurgitation indicated for surgical placement of a RVOT conduit of bioprosthetic valve. The imaging data and model for development of the transcatheter pulmonary valve can be obtained in a clinical study. A clinical study for use of a heart valve for RVOT anomaly can be defined as follows: (i) limited study on a device early in development, before the design is finalized (information gained to guide device modification); and (ii) evaluate device design concept (safety and functionality) in a small number of patients when information cannot be obtained through other non-clinical tests (animal or bench). Early feasibility studies can be used to characterize device/anatomic boundary conditions (collect human data) for input to device development and testing. Animal models have proved insufficient to characterize device/anatomic boundary conditions because no animal model even closely simulates human pulmonic/RVOT anatomy or loading conditions. Data from existing products and literature may be leveraged to some extent in lieu of testing, which can be the case regardless of the device, conduit, and disease to be treated.

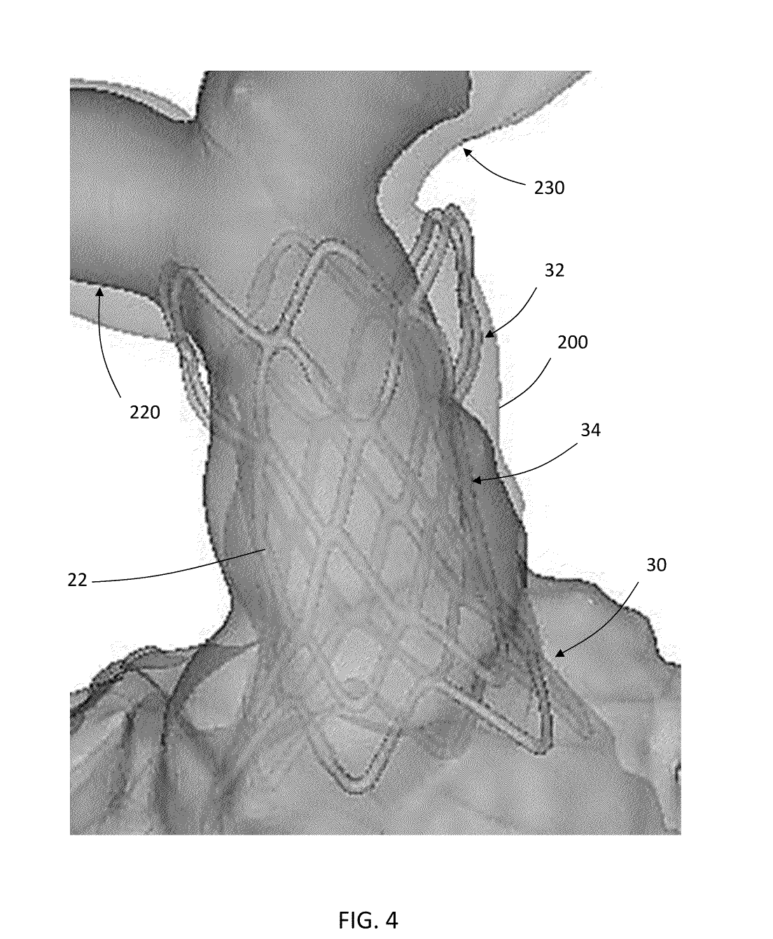

Referring now to FIG. 4, a virtual image right heart model showing a pulmonary artery 200 is overlaid on a virtual image of a fully expanded frame 22 of a device, such as a prosthetic heart valve. The model includes minimum dimensional characteristics 220 shown in darker shading and maximum dimensional characteristics 230 shown in lighter shading. At least portions of the outflow region 32 or distal region of the fully expanded frame 22 have a diametric dimension or perimeter greater than the diametric dimension or perimeter of the pulmonary artery 200 at a location along the length of the artery 200 at which the frame 22 may be implanted. Such a relationship will allow the outflow region 32 of the frame 22 to retain the frame's location within the artery 200 when implanted via interference fit. However, the minimum diametric dimension of the artery 200 should not be below a limit at which the frame 22 will collapse and no longer be biased towards its fully expanded state. The dimensional characteristic of the expanded frame 22 in FIG. 4 in a mid-region 34 is about the same as, or less than, the minimum dimensional characteristic of the artery 200 along a length of the artery in which the mid-region 34 of the frame 22 is to be implanted. In the embodiment depicted in FIG. 4, the inflow (proximal) region 30 of the frame 22 has a fully expanded diametric dimension or perimeter that is about the same as the maximum diametric dimension or perimeter of a region of the pulmonary artery 200 in which it would be implanted. Preferably, the inflow (proximal) region 30 of the frame 22 has a fully expanded diametric dimension or perimeter that is greater than the maximum diametric dimension or perimeter of a region of the pulmonary artery 200 in which it would be implanted to allow the inflow region 30 of the frame 22 to retain the frame's location within the artery 200 when implanted via interference fit.

Regardless of the type of implantable expandable medical device or the conduit into which the device is to be implanted, in various embodiments disclosed herein the expandable implantable device is selected or built to have a maximum dimensional characteristic enabling an interference fit within the conduit when the conduit, or portions thereof, are at maximum diametric or perimeter dimensions. Accordingly, the maximum dimensional characteristic of at least a portion of the implantable device, along the length of the implantable device, may be larger than the maximum dimensional characteristic of the conduit at a location in which the portion of the device is to be implanted. In some embodiments, the maximum dimensional characteristic of the conduit at a predetermined location along the length of the conduit is in the range from about 60% to about 95% of the maximum dimensional characteristic of the implantable medical device at a location along a distal portion of the medical device having the largest dimensional characteristic of the distal portion of the medical device. In some embodiments, the maximum dimensional characteristic of the conduit at a predetermined location along the length of the conduit is about 85% or less, such as about 80% or less, than the maximum dimensional characteristic of the implantable medical device at a location along a distal portion of the medical device having the largest dimensional characteristic of the distal portion of the medical device.

In some embodiments, the implantable medical device is a prosthetic heart valve having an expandable frame. In some embodiments, the prosthetic heart valve is a prosthetic pulmonary valve (also referred to as a pulmonic valve). In various embodiments, the maximum dimensional characteristic of the conduit at a predetermined location along the length of the conduit is in a range from about 60% to about 95% of the maximum dimensional characteristic of the medical device at a location along a distal (outflow) portion of the prosthetic heart valve having the largest dimensional characteristic of the distal portion of the prosthetic heart valve. In some embodiments. In some embodiments, the maximum dimensional characteristic of the conduit at a predetermined location along the length of the conduit is about 85% or less, such as about 80% or less, than the maximum dimensional characteristic of the medical device at a location along a distal portion of the prosthetic heart valve having the largest dimensional characteristic of the distal portion of the prosthetic heart valve. In some embodiments, the maximum dimensional characteristic of the conduit at a predetermined location along the length of the conduit is in a range from about 60% to about 90% of the maximum dimensional characteristic of the prosthetic heart valve at a location along a proximal (inflow) portion of the prosthetic heart valve having the largest dimensional characteristic of the proximal portion of the prosthetic heart valve. In some embodiments, the maximum dimensional characteristic of the conduit at a predetermined location along the length of the conduit is about 85% or less, such as about 80% or less, than the maximum dimensional characteristic of the medical device at a location along a proximal portion of the prosthetic heart valve having the largest dimensional characteristic of the proximal portion of the prosthetic heart valve.

FIG. 5 is a plot showing dimensions of a prosthetic pulmonary valve device along the length of the prosthetic valve device (the device is depicted to the right of the plot), dimensions of a portion of a conduit (in this case, the pulmonary artery) along a length of a portion of the conduit into which the prosthetic valve may be implanted, and a desired region in which the perimeters of the conduit preferably fall over the entire cardiac cycle for the given heart valve. The triangular shaded areas to the right of the vertical (minimum perimeter) line indicate a preferred range of perimeters along the length of a portion of the conduit. If the perimeters of the conduit along the length of the conduit fall within the preferred areas over the cardiac cycle (or an acceptable portion thereof), the device will likely properly fit that portion of the conduit and properly function within that portion of the conduit. Further shown in FIG. 5 are traces of perimeter values along the length of the conduit at phase=30% of the cardiac cycle and phase=90% of the cardiac cycle, which were determined to be phases at which maximum and minimum pulmonary artery perimeters may be observed. A trace of the perimeter value of the fully expanded device over the length of the device is also depicted. As shown, the preferred area (shaded area in FIG. 5) has smaller perimeters (over at least a portion of the length) than the fully expanded device perimeter to provide for interference fit of the device within the conduit. The device traces depicted in FIG. 5 may indicate that the device is appropriate for implantation in the given location of the conduit from which the traces were obtained. However, it is noted that there would likely be a loss of contact of the proximal portion of the device in diastole (phase=90%), a potential for too great a valve constraint in the mid-section, and the conduit portion may be too short for the device in systole (phase=30%). As indicated, a great deal of information may be obtained by comparing device dimensional characteristics with dimensional characteristics of a conduit into which the device is to be implanted for purposes of one or more of (i) determining whether the device may fit into the location of the conduit, (ii) identifying which of a plurality of devices may be suitable for implanting in the selected location, (iii) identifying a region of the conduit into which a given device may properly fit, (iv) manufacturing a custom device for implanting in the selected location, (vi) manufacturing a family of devices having specified dimensional characteristics, and (v) the like.

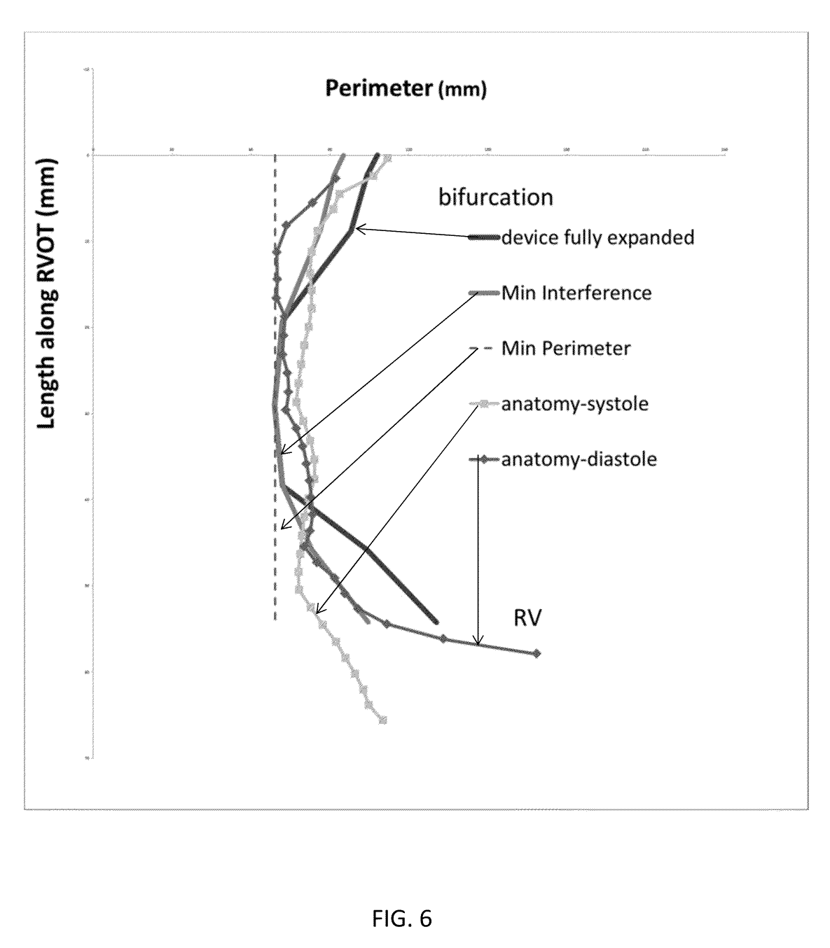

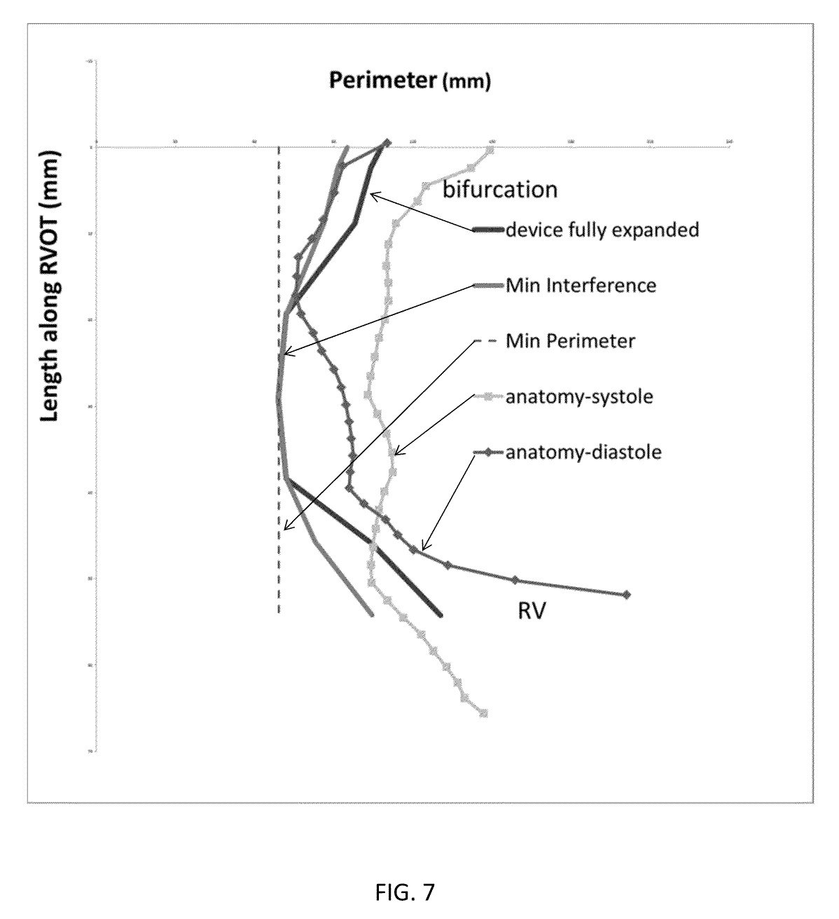

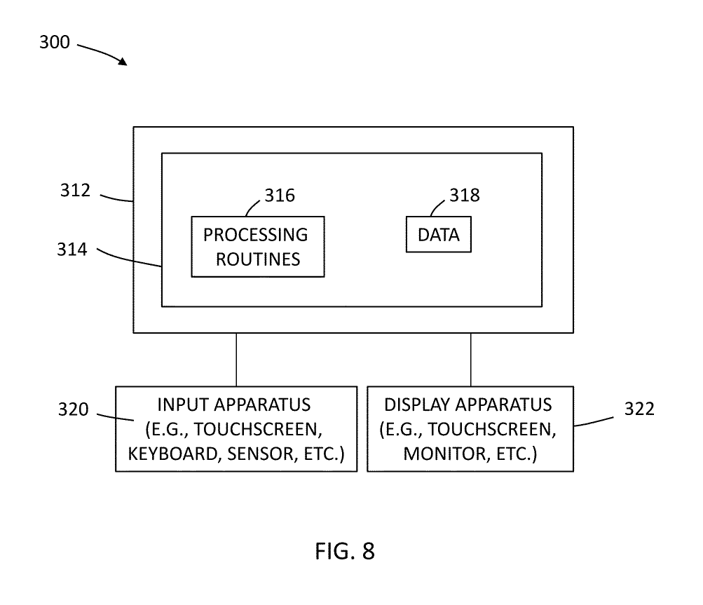

FIGS. 6-7 show additional traces of portions of conduits in terms of perimeter values over the length of the portion of the conduit at maximum and minimum conduit perimeters compared with perimeters of a potential device for implantation in the conduit at the selected portions of the conduits. It is noted that the device trace compared to the conduit traces indicate a better fit in FIG. 6 than in FIG. 7.

A schematic drawing of an embodiment of a system 300 depicted in FIG. 8 may be used to execute embodiments of the methods or processes described herein. The system 300 could, for example, include a desktop computer, a laptop computer, a tablet device, or the like.

In the embodiment depicted in FIG. 8, the system 300 includes computing apparatus 312. The computing apparatus 312 may be configured to receive input from input apparatus 320 and transmit output to display apparatus 322. Further, the computing apparatus 312 may include data storage 314. Data storage 314 may allow for access to processing programs or routines 316 and one or more other types of data 318 that may be employed to carry out the methods or processes described herein. For example, the computing apparatus 312 may be configured to calculate dimensional characteristics of a conduit from imaging data, determine fit of a medical device in the conduit based on the imaging data, and display status information on the regarding the dimensions, fit, or the like on display apparatus 322.

The computing apparatus 312 may be operatively coupled to the input apparatus 320 and the display apparatus 322 to, e.g., transmit data to and from each of the input apparatus 320 and the display apparatus 322. For example, the computing apparatus 312 may be electrically coupled to each of the input apparatus 320 and the display apparatus 322 using, e.g., analog electrical connections, digital electrical connections, wireless connections, bus-based connections, etc. As described further herein, a user may provide input to the input apparatus 320 to manipulate, or modify, one or more graphical depictions displayed on the display apparatus 322.

Further, various devices and apparatuses may be operatively coupled to the computing apparatus 312 to be used with the computing apparatus 312 to perform one or more of the methods, processes or logic described herein. As shown, the system 300 may include input apparatus 320, display apparatus 322. If the system 300 includes imaging apparatus (not shown in FIG. 8), the imaging apparatus may be operably coupled to the computing apparatus 312.

The input apparatus 320 may include any apparatus capable of providing input to the computing apparatus 312 to perform the functionality, methods, processes or logic described herein. For example, the input apparatus 320 may include a touchscreen (e.g., capacitive touchscreen, a resistive touchscreen, a multi-touch touchscreen, etc.), a mouse, a keyboard, a trackball, hematocrit sensor, etc. The input apparatus 320 may allow a user to select and view various status information corresponding to dimensions of a conduit or a device or fit of the device within the conduit when used in conjunction with the display apparatus 322 (e.g., displaying a graphical user interface).

Likewise, the display apparatus 322 may include any apparatus capable of displaying information to a user, such as a graphical user interface, etc., to perform the functionality, methods, processes or logic described herein. For example, the display apparatus 322 may include a liquid crystal display, an organic light-emitting diode screen, a touchscreen, a cathode ray tube display, etc. As described further herein, the display apparatus 322 may be configured to display a graphical user interface that includes one or more regions corresponding to one or more parameters of conduit dimensions, device dimensions, fit of device in conduit, etc. As used herein, a “region” of a graphical user interface may be defined as a portion of the graphical user interface within which information may be displayed or functionality may be performed. Regions may exist within other regions, which may be displayed separately or simultaneously. For example, smaller regions may be located within larger regions, regions may be located side-by-side, etc. Additionally, as used herein, an “area” of a graphical user interface may be defined as a portion of the graphical user interface located with a region that is smaller than the region it is located within.

The processing programs or routines 316 may include programs or routines for performing computational mathematics, matrix mathematics, standardization algorithms, comparison algorithms, or any other processing required to implement one or more methods or processes, or portions or combinations thereof, described herein. Data 318 may include, for example, imaging data (raw or preprocessed), device dimension data, notifications, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein, or any other data that may be necessary for carrying out one more processes or methods, or portions or combinations thereof, described herein.

In one or more embodiments, the system 300 may be implemented using one or more computer programs executed on programmable computers, such as computers that include, for example, processing capabilities, data storage (e.g., volatile or non-volatile memory or storage elements), input devices, and output devices. Program code or logic described herein may be applied to input data to perform functionality described herein and generate desired output information. The output information may be applied as input to one or more other devices or methods as described herein or as would be applied in a known fashion.

The program used to implement methods or processes, or portions or combinations thereof, described herein may be provided using any programmable language, e.g., a high level procedural or object orientated programming language that is suitable for communicating with a computer system. Any such programs may, for example, be stored on any suitable device, e.g., a storage media, that is readable by a general or special purpose program running on a computer system (e.g., including processing apparatus) for configuring and operating the computer system when the suitable device is read for performing the procedures described herein. In other words, in embodiments, the system 300 may be implemented using a computer readable storage medium, configured with a computer program, where the storage medium so configured causes the computer to operate in a specific and predefined manner to perform functions described herein. Further, in embodiments, the system 200 may be described as being implemented by logic (e.g., object code) encoded in one or more non-transitory media that includes code for execution and, when executed by a processor, is operable to perform operations such as the methods, processes, or functionality, or portions or combinations thereof, described herein.

Likewise, the system 300 may be configured at a remote site (e.g., an application server) that allows access by one or more users via a remote computer apparatus (e.g., via a web browser), and allows a user to employ the functionality according to the present disclosure (e.g., user accesses a graphical user interface associated with one or more programs to process data).

The computing apparatus 312 may be, for example, any fixed or mobile computer system (e.g., a controller, a microcontroller, a personal computer, mini computer, etc.). The exact configuration of the computing apparatus 312 is not limiting, and essentially any device capable of providing suitable computing capabilities (e.g., graphics processing, etc.) may be used.

As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by computing apparatus 312 described herein.

Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes or programs (e.g., the functionality provided by such systems, processes or programs) described herein.

One will recognize that graphical user interfaces may be used in conjunction with the embodiments described herein. The graphical user interfaces may provide various features allowing for user input thereto, change of input, importation or exportation of files, or any other features that may be generally suitable for use with the processes described herein. For example, the graphical user interfaces may allow default values to be used or may require entry of certain values, limits, threshold values, or other pertinent information.

The methods, processes, functionality or logic, or portions or combinations thereof, described in this disclosure, including those attributed to the systems, or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, or other devices. The term “processor” or “processing circuitry” may generally refer to any of the foregoing logic circuitry, alone or in combination with other logic circuitry, or any other equivalent circuitry.

Such hardware, software, or firmware may be implemented within the same device or within separate devices to support the various operations and functions described in this disclosure. In addition, any of the described components may be implemented together or separately as discrete but interoperable logic devices. Depiction of different features, e.g., using block diagrams, etc., is intended to highlight different functional aspects and does not necessarily imply that such features must be realized by separate hardware or software components. Rather, functionality may be performed by separate hardware or software components, or integrated within common or separate hardware or software components.

When implemented in software, the functionality ascribed to the systems, devices and methods described in this disclosure may be embodied as instructions and/or logic on a computer-readable medium such as RAM, ROM, NVRAM, EEPROM, FLASH memory, magnetic data storage media, optical data storage media, or the like. The instructions and/or logic may be executed by one or more processors to support one or more aspects of the functionality described in this disclosure.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. For example, a method that “comprises” steps A, B, and C may be a method that “consists of” steps A, B and C or that “consists essentially of” steps A, B and C.

As used herein, “consisting essentially of,” as it relates to a composition, apparatus, system, method or the like, means that the components of the composition, apparatus, system, method or the like are limited to the enumerated components and any other components that do not materially affect the basic and novel characteristic(s) of the composition, apparatus, system, method or the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

A number of aspects of methods, devices, and systems have been described herein. A summary of some selected aspects is presented below.

A first aspect is a non-invasive method for determining whether an expandable medical device is appropriate for implanting in a conduit of patient, comprising: (A) imaging a portion of the conduit of the patient into which the medical device is to be implanted, wherein the imaging is performed during a plurality of portions of the cardiac cycle, such as (i) a portion of the cardiac cycle at which one or more dimensional characteristics of the conduit are at or near minimums and (ii) a portion of the cardiac cycle at which the one or more dimensional characteristics of the conduit are at or near maximums; (B) deriving, from the imaging, minimum and maximum dimensional characteristics of the conduit at a plurality of locations along a length of the portion of the conduit; and (C) determining that the medical device is appropriate for implantation if the maximum and minimum dimensional characteristics of the conduit at the predetermined locations along the length of the conduit are within predetermined ranges of maximum, expanded and minimum, contracted dimensional characteristic of the medical device at predetermined locations along the length of the medical device (e.g., to determine predetermined ranges of interference fit).

A second aspect is a method according to the first aspect wherein the medical device is a transcatheter medical device.

A third aspect is a method according to the first or second aspect, wherein the medical device comprises a prosthetic heart valve.

A fourth aspect is a method according to any one of the preceding aspects, wherein the imaging comprises magnetic resonance imaging, ultrasound imaging, computed tomography imaging, or x-ray imaging.

A fifth aspect is a method according to any one of the preceding aspects, wherein imaging comprises capturing image data at or near 30% phase and at or near 90% phase of a cardiac cycle of the patient.

A sixth aspect is a method according to any one of the preceding aspects, wherein the maximum dimensional characteristic of the conduit at a predetermined location along the length of the conduit is 60% to 95% of the maximum dimensional characteristic of the medical device at a location along a distal portion of the medical device having the largest dimensional characteristic of the distal portion of the medical device.

A seventh aspect is a method according to any one of the preceding aspects, wherein the maximum dimensional characteristic of the conduit at a predetermined location along the length of the conduit is 85% or less than the maximum dimensional characteristic of the medical device at a location along a distal portion of the medical device having the largest dimensional characteristic of the distal portion of the medical device.

An eighth aspect is a method according to any one of the preceding aspects, wherein the maximum dimensional characteristic of the conduit at a predetermined location along the length of the conduit is 60% to 90% of the maximum dimensional characteristic of the medical device at a location along a proximal portion of the medical device having the largest dimensional characteristic of the proximal portion of the medical device.

A ninth aspect is a method according to any one of the preceding aspects, wherein the maximum dimensional characteristic of the conduit at a predetermined location along the length of the conduit is 85% or less than the maximum dimensional characteristic of the medical device at a location along a proximal portion of the medical device having the proximal dimensional characteristic of the distal portion of the medical device.

A tenth aspect is a method according to any one of the preceding aspects, wherein the maximum dimensional characteristic of the conduit at a predetermined location along the length of the conduit is 80% or less than the maximum dimensional characteristic of the medical device at a location along a proximal portion of the medical device having the proximal dimensional characteristic of the distal portion of the medical device.

An eleventh aspect is a method according to any one of the preceding aspects, wherein the method is a computer-implemented method.

A twelfth aspect is a computer implemented method for determining whether an expandable medical device is appropriate for implanting in a conduit of patient, comprising: (A) receiving input of imaging data regarding a portion of the conduit of the patient into which the medical device is to be implanted, wherein the imaging data comprises data obtained during a plurality of portions of the cardiac cycle, such as (i) a portion of the cardiac cycle at which dimensional characteristics of the conduit are at or near minimums and (ii) a portion of the cardiac cycle at which the dimensional characteristics of the conduit are at or near maximums; (B) calculating minimum and maximum dimensional characteristics of the conduit at a plurality of locations along a length of the portion of the conduit based on the imaging data; and (C) determining whether the maximum and minimum dimensional characteristics of the conduit at the predetermined locations along the length of the conduit are within predetermined ranges of maximum, expanded and minimum, contracted dimensional characteristics of the medical at predetermined locations along the length of the medical.

A thirteenth aspect is a method according to the twelfth aspect, wherein the medical device is a transcatheter medical device.

A fourteenth aspect is a method according to the twelfth or thirteenth aspect, wherein the medical device comprises a prosthetic heart valve.

A fifteenth aspect is a method according to any one of aspects 12-14, further comprising receiving input regarding the length and maximum, expanded and minimum, contracted dimensional characteristics of the medical device at predetermined locations along the length of the medical device.

A sixteenth aspect is a method according to any one of aspects 12-15, further comprising displaying data indicative of whether the medical device is appropriate for implanting in the conduit of the patient.

A seventeenth aspect is a non-transitory computer-readable medium that when executed by a device causes the device to carry out a method according to any one of aspects 12-16.

An eighteenth aspect is a device for determining whether an expandable medical device is appropriate for implanting in a conduit of patient, the device comprising: (A) input apparatus configured to receive input of imaging data regarding a portion of the conduit of the patient into which the medical device is to be implanted, wherein the imaging data comprises data obtained during a plurality of portions of the cardiac cycle, such as (i) a portion of the cardiac cycle at which dimensional characteristics of the conduit are at or near minimums and (ii) a portion of the cardiac cycle at which the dimensional characteristics of the conduit are at or near maximums; and (B) computing apparatus configured to: (i) calculate minimum and maximum dimensional characteristics of the conduit at a plurality of locations along a length of the portion of the conduit based on the imaging data; and (ii) determining whether the maximum and minimum dimensional characteristics of the conduit at the predetermined locations along the length of the conduit are within predetermined ranges of maximum, expanded and minimum, contracted dimensional characteristics of the medical device at predetermined locations along the length of the medical device.

A nineteenth aspect is a device according to the eighteenth aspect, wherein the medical device is a transcatheter medical device.

A twentieth aspect is a device according to the eighteenth or nineteenth aspect, wherein the medical device comprises a prosthetic heart valve.

A twenty-first aspect is a device according to any one of aspects 18-20, wherein the input apparatus is further configured to receive input regarding the length and maximum, expanded and minimum, contracted dimensional characteristics of the medical at predetermined locations along the length of the medical device.

A twenty-second aspect is a device according to any one of aspects 18-21, further comprising a display operably coupled to the control electronics and configured to display data regarding whether the medical device is appropriate for implanting in a conduit of the patient.

A twenty-third aspect is a non-invasive method for selecting an expandable medical device for implanting in a conduit of a patient, comprising: (A) imaging a portion of the conduit of the patient into which the medical device is to be implanted, wherein the imaging is performed during a plurality of portions of the cardiac cycle, such as (i) a portion of the cardiac cycle at which the dimensional characteristics of the conduit are at or near minimums and (ii) a portion of the cardiac cycle at which the dimensional characteristics of the conduit are at or near maximums; (B) deriving, from the imaging, minimum and maximum dimensional characteristics of the conduit at a plurality of locations along a length of the portion of the conduit; and (C) selecting a medical device as appropriate for implanting in the patient if the maximum and minimum dimensional characteristics of the conduit at the predetermined locations along the length of the conduit are within predetermined ranges of maximum, expanded and minimum, contracted dimensional characteristics of the medical at predetermined locations along the length of the medical device.

A twenty-fourth aspect is a method according to the twenty-third aspect, wherein the medical device is a transcatheter medical device.

A twenty-fifth aspect is a method according to the twenty-third or twenty-fourth aspect, wherein the medical device comprises a prosthetic heart valve.

A twenty-sixth aspect is a method according to any one of aspects 23-25, wherein the imaging comprises magnetic resonance imaging, ultrasound imaging, computed tomography imaging, or x-ray imaging.

A twenty-seventh aspect is a method according to any one of aspects 23-26, wherein imaging comprises capturing image data at or near 30% phase and at or near 90% phase of a cardiac cycle of the patient.

A twenty-eights aspect is a method according to any one aspects 23-27, wherein maximum dimensional characteristic of the conduit at a predetermined location along the length of the conduit is 60% to 95% of the maximum dimensional characteristic of the medical device at a location along a distal portion of the medical device having the largest dimensional characteristic of the distal portion of the medical device.

A twenty-ninth aspect is a method according to any one of aspects 23-28, wherein the maximum dimensional characteristic of the conduit at a predetermined location along the length of the conduit is 85% or less than the maximum dimensional characteristic of the medical device at a location along a distal portion of the medical device having the largest dimensional characteristic of the distal portion of the medical device.

A thirtieth aspect is a method according to any one of aspects 23-29, wherein the maximum dimensional characteristic of the conduit at a predetermined location along the length of the conduit is 60% to 90% of the maximum dimensional characteristic of the medical device at a location along a proximal portion of the medical device having the largest dimensional characteristic of the proximal portion of the medical device.

A thirty-first aspect is a method according to any one of aspects 23-30, wherein the maximum dimensional characteristic of the conduit at a predetermined location along the length of the conduit is 85% or less that the maximum dimensional characteristic of the medical device at a location along a proximal portion of the medical device having the proximal dimensional characteristic of the distal portion of the medical device.

A thirty-second aspect is a method according to any one aspects 23-31, wherein the maximum dimensional characteristic of the conduit at a predetermined location along the length of the conduit is 80% or less than the maximum dimensional characteristic of the device at a location along a proximal portion of the medical device having the proximal dimensional characteristic of the distal portion of the medical device.

A thirty-third aspect is a method according to any one aspects 23-32, wherein the method is a computer-implemented method.

A thirty-fourth aspect is a computer implemented method for selecting an expandable medical device for implanting in a conduit of a patient, comprising: (A) receiving input of imaging data regarding a portion of the conduit of the patient into which the medical device is to be implanted, wherein the imaging data comprises data obtained during a plurality of portions of the cardiac cycle, such as (i) a portion of the cardiac cycle at which dimensional characteristics of the conduit are at or near minimums and (ii) a portion of the cardiac cycle at which the dimensional characteristics of the conduit are at or near maximums; (B) calculating minimum and maximum dimensional characteristics of the conduit at a plurality of locations along a length of the portion of the conduit based on the imaging data; and (C) comparing the maximum and minimum dimensional characteristics of the conduit at the predetermined locations along the length of the conduit to parameters of a plurality of medical devices, wherein the parameters of the medical devices comprise lengths and maximum, expanded and minimum, contracted dimensional characteristics of the medical devices at predetermined locations along the lengths of the medical devices; and (D) determining whether one or more of the plurality of medical devices is appropriate for implanting in the patient if the maximum and minimum dimensional characteristics of the conduit at the predetermined locations along the length of the conduit are within predetermined ranges of maximum, expanded and minimum, contracted dimensional characteristics of the one or more medical devices at predetermined locations along the lengths of the medical devices.

A thirty-fifth aspect is a method according to the thirty-fourth aspect, wherein the medical devices are trhanscatheter medical devices.

A thirty-sixth aspect is a method according to the thirty-fourth or thirty-fifth aspect, wherein the medical devices comprise prosthetic heart valves.

A thirty-seventh aspect is a method according to any one of aspects 34-46, further comprising determining which of the one or more appropriate medical devices is most suitable for implanting in the patient by determining which of the one or more medical devices has maximum, expanded dimensional characteristics at predetermined locations along the length of the medical device that deviate the least from predetermined preferred percentages of the maximum dimensional characteristics of the portion of the conduit at the predetermined locations along the length of the portion of the conduit.

A thirty-eighth aspect is a method according to the thirty-seventh aspect, wherein determining which of the one or more appropriate medical devices is most suitable for implanting in the patient based on the patient's anatomy comprises applying weighing factors to a plurality of the maximum dimensional characteristics at the predetermined locations along the length of the portion of the conduit.

A thirty-ninth aspect is a method according to any one of aspects 34-38, further comprising receiving input regarding the lengths and maximum, expanded and minimum, contracted dimensional characteristics of the medical device at the predetermined locations along the lengths of the plurality of medical devices.

A fortieth aspect is a method according to any one of aspects 34-39, further comprising displaying data indicative of which of the plurality of medical devices is appropriate for implanting in the conduit of the patient.

A forty-first aspect is a non-transitory computer-readable medium that when executed by a device causes the device to carry out a method according to any one of aspects 34-40.

A forty-second aspect is a device for determining whether an expandable medical device is appropriate for implanting in a conduit of patient, the device comprising: (A) input apparatus configured to receive input of imaging data regarding a portion of the conduit of the patient into which the medical device is to be implanted, wherein the imaging data comprises data obtained during a plurality of portions of the cardiac cycle, such as (i) a portion of the cardiac cycle at which dimensional characteristics of the conduit are at or near minimums and (ii) a portion of the cardiac cycle at which the dimensional characteristics of the conduit are at or near maximums; and (B) computing apparatus configured to: (i) calculate minimum and maximum dimensional characteristics of the conduit at a plurality of locations along a length of the portion of the conduit based on the imaging data; and (ii) compare the maximum and minimum dimensional characteristics of the conduit at the predetermined locations along the length of the conduit to parameters of a plurality of medical devices, wherein the parameters of the medical devices comprise lengths and maximum, expanded and minimum, contracted dimensional characteristics of the medical devices at predetermined locations along the lengths of the medical devices; and (C) determine whether one or more of the plurality of medical devices to be appropriate for implanting in the patient if the maximum and minimum dimensional characteristics of the conduit at the predetermined locations along the length of the conduit are within predetermined ranges of maximum, expanded and minimum, contracted dimensional characteristics of the one or more medical devices at predetermined locations along the lengths of the medical devices.

A forty-third aspect is a device according to the forty-second aspect, wherein the medical devices are transcatheter medical devices.

A forty-fourth aspect is a device according to the forty-second or forty-third aspects, wherein the medical devices comprise heart valves.

A forty-fifth aspect is a device according to any one of aspects 42-44, wherein the input apparatus is further configured to receive input regarding the lengths and maximum, expanded and minimum, contracted dimensional characteristics of the plurality of medical devices at the predetermined locations along the length of the medical devices.

A forty-sixth aspect is a device according to any one of aspects 42-45, further comprising a display operably coupled to the control electronics and configured to display data regarding which of the plurality of medical devices is appropriate for implanting in the conduit of the patient.

A forty-seventh aspect is a non-invasive method for selecting an appropriate region of a conduit of a patient for implanting an expandable medical device, comprising: (A) imaging a portion of the conduit of the patient into which the medical device is to be implanted, wherein the imaging is performed during a plurality of portions of the cardiac cycle, such as (i) a portion of the cardiac cycle at which the dimensional characteristics of the conduit are at or near minimums and (ii) a portion of the cardiac cycle at which the dimensional characteristics of the conduit are at or near maximums; (B) deriving, from the imaging, minimum and maximum dimensional characteristics of the conduit at a plurality of locations along a length of the portion of the conduit; and (C) determining whether a region in the portion of the conduit has maximum and minimum dimensional characteristics at predetermined distances along the length of the conduit in the region that are within predetermined ranges of maximum, expanded and minimum, contracted dimensional characteristics of the medical device at predetermined locations along the length of the medical device.

A forty-eighth aspect is a method according to the forty-seventh aspect, wherein the medical device is a transcatheter medical device.

A forty-ninth aspect is a method according to the forty-seventh or forty-eighth aspect, wherein the medical device comprises a prosthetic heart valve.

A fiftieth aspect is a method according to any one of aspects 47-49, further comprising selecting the region of the conduit as appropriate for implanting the medical device if the maximum and minimum dimensional characteristics of the conduit at the predetermined locations along the length of the conduit in the region are within predetermined ranges of maximum, expanded and minimum, contracted dimensional characteristics of the medical device at predetermined locations along the length of the medical device.

A fifty-first aspect is a method according to any one of aspects 47-50, wherein the imaging comprises magnetic resonance imaging, ultrasound imaging, computed tomography imaging, or x-ray imaging.

A fifty-second aspect is a method according to any one of aspects 47-51, wherein imaging comprises capturing image data at or near 30% phase and at or near 90% phase of a cardiac cycle of the patient.

A fifty-third aspect is a method according to any one aspects 47-52, wherein the maximum dimensional characteristic of the conduit at a predetermined location along the length of the conduit is 60% to 95% of the maximum dimensional characteristic of the medical device at a location along a distal portion of the medical device having the largest dimensional characteristic of the distal portion of the medical device.

A fifty-fourth aspect is a method according to any one of aspects 47-53, wherein the maximum dimensional characteristic of the conduit at a predetermined location along the length of the conduit is 85% or less than the maximum dimensional characteristic of the medical device at a location along a distal portion of the medical device having the largest dimensional characteristic of the distal portion of the medical device.

A fifty-fifth aspect is a method according to any one of aspects 47-54, wherein the maximum dimensional characteristic of the conduit at a predetermined location along the length of the conduit is 60% to 90% of the maximum dimensional characteristic of the medical device at a location along a proximal portion of the medical device having the largest dimensional characteristic of the proximal portion of the medical device.

A fifty-sixth aspect is a method according to any one of aspects 47-55, wherein the maximum dimensional characteristic of the conduit at a predetermined location along the length of the conduit is 85% or less than the maximum dimensional characteristic of the medical device at a location along a proximal portion of the medical device having the proximal dimensional characteristic of the distal portion of the medical device.

A fifty-seventh aspect is a method according to any one aspects 47-56, wherein the maximum dimensional characteristic of the conduit at a predetermined location along the length of the conduit is 80% or less than the maximum dimensional characteristic of the medical device at a location along a proximal portion of the medical device having the proximal dimensional characteristic of the distal portion of the medical device.

A fifty-eighth aspect is a method according to any one aspects 47-57, wherein the method is a computer-implemented method.

A fifty-ninth aspect is a computer implemented method for selecting an appropriate region of a conduit of a patient for implanting an expandable medical device, comprising: (A) receiving input of imaging data regarding a portion of the conduit of the patient into which the medical device is to be implanted, wherein the imaging data comprises data obtained during a plurality of portions of the cardiac cycle, such as (i) a portion of the cardiac cycle at which dimensional characteristics of the conduit are at or near minimums and (ii) a portion of the cardiac cycle at which the dimensional characteristics of the conduit are at or near maximums; (B) calculating minimum and maximum dimensional characteristics of the conduit at a plurality of locations along a length of the portion of the conduit based on the imaging data; and (C) comparing the maximum and minimum dimensional characteristics of the conduit at the predetermined locations along the length of the conduit to maximum, expanded and minimum, contracted dimensional characteristics of the medical device at predetermined locations along the length of the medical device; and (D) determining whether a region in the portion of the conduit has maximum and minimum dimensional characteristics at predetermined distances along the length of the conduit in the region that are within predetermined ranges of maximum, expanded and minimum, contracted dimensional characteristics of the medical device at predetermined locations along the length of the medical device.

A sixtieth aspect is a method according to the fifty-ninth aspect, wherein the medical device is a transcatheter medical device.

A sixty-first aspect is a method according to the fifty-ninth or sixtieth aspect, wherein the medical device comprises a heart valve.

A sixty-second aspect is a method according to any one of aspects 59-61, further comprising determining which region of one or more regions within the portion of the conduit is most suitable for implanting the medical device by determining which of the one or more regions has maximum dimensional characteristics along the length of the region that deviate the least from predetermined preferred percentages of the maximum, expanded dimensional characteristics at predetermined locations along the length of the medical device.

A sixty-third aspect is a method according to the sixty-second aspect, wherein determining which of the one or more regions is most suitable for implanting the medical device comprises applying weighing factors to a plurality of the maximum dimensional characteristics at the predetermined locations along the length of the portion of the conduit.

A sixty-fourth aspect is a method according to any one of aspects 59-63, further comprising receiving input regarding the length and maximum, expanded and minimum, contracted dimensional characteristic of the medical device at the predetermined locations along the length of the medical device.

A sixty-fifth aspect is a method according to any one of aspects 59-64, further comprising displaying data indicative of whether the region of the portion of the conduit is appropriate for implanting the medical device.

A sixty-sixth aspect is a non-transitory computer-readable medium that when executed by a device causes the device to carry out a method according to any one of aspects 59-65.

A sixty seventh aspect is a device for selecting an appropriate region of a conduit of a patient for implanting an expandable medical device, the device comprising: (A) input apparatus configured to receive input of imaging data regarding a portion of the conduit of the patient into which the medical device is to be implanted, wherein the imaging data comprises data obtained during a plurality of portions of the cardiac cycle, such as (i) a portion of the cardiac cycle at which dimensional characteristics of the conduit are at or near minimums and (ii) a portion of the cardiac cycle at which the dimensional characteristics of the conduit are at or near maximums; and (B) computing apparatus configured to: (i) calculate minimum and maximum dimensional characteristics of the conduit at a plurality of locations along a length of the portion of the conduit based on the imaging data; and (ii) compare the maximum and minimum dimensional characteristics of the conduit at the predetermined locations along the length of the conduit to maximum, expanded and minimum, contracted dimensional characteristics of the medical devices at predetermined locations along the length of the medical device; and (C) determine whether a region in the portion of the conduit has maximum and minimum dimensional characteristics at predetermined distances along the length of the conduit in the region that are within predetermined ranges of maximum, expanded and minimum, contracted dimensional characteristics of the medical device at predetermined locations along the length of the medical device.

A sixty-eighth aspect is a device according to the sixty-seventh aspect, wherein the medical device is a transcatheter medical device.

A sixty-ninth aspect is a device according to the sixty-seventh or sixty-eighth aspect, wherein the medical device comprises a prosthetic heart valve.

A seventieth aspect is a device according to any one of aspects 67-69, wherein the input apparatus is further configured to receive input regarding the length and maximum, expanded and minimum, contracted dimensional characteristic of the medical device at the predetermined locations along the length of the medical device.

A seventy-first aspect is a device according to any one of aspects 67-70, further comprising a display operably coupled to the control electronics and configured to display data whether a region within the portion of the conduit is appropriate for implanting the medical device.

A seventy-second aspect is a method for determining appropriate length and diametric specifications for an expandable medical device for implanting in a conduit of a patient, comprising: (A) imaging a portion of the conduit of the patient into which the medical device is to be implanted, wherein the imaging is performed during a plurality of portions of the cardiac cycle, such as (i) a portion of the cardiac cycle at which the dimensional characteristics of the conduit are at or near minimums and (ii) a portion of the cardiac cycle at which the dimensional characteristics of the conduit are at or near maximums; (B) deriving, from the imaging, minimum and maximum dimensional characteristics of the conduit at a plurality of locations along a length of the portion of the conduit; and (C) determining the appropriate length and minimum, contracted and maximum, expanded dimensional characteristics at various locations along the length of the medical device as a length and dimensional characteristics that are at predetermined preferred maximum and minimum dimensional characteristics at the plurality of locations along the length of the conduit.

A seventy-third aspect is a method according to the seventy-second aspect, further comprising manufacturing an expandable medical device to have the determined appropriate length and minimum, contracted and maximum, expanded dimensional characteristics.

A seventy-fourth aspect is a method according to the seventy-second or seventy-third aspect, wherein the medical device is a transcatheter medical device.

A seventy-fifth aspect is a method according to any one of aspects 72-74, wherein the medical device comprises a heart valve.

A seventy-sixth aspect is a method or device according to any one of the preceding aspects, wherein the conduit is a bioprosthetic conduit or device.

A seventy-seventh aspect is a method or device according to any one of preceding aspects, wherein the conduit is a heart, vein or artery.

A seventy-eighth aspect is a method or device according to any one of the preceding aspects, wherein at least one of the one or more dimensional characteristic is a perimeter, diameter, area, or length.

Thus, embodiments of NON-INVASIVE EVALUATION OF FIT AND SIZING OF DEVICE PRIOR TO IMPLANT are disclosed. One skilled in the art will appreciate that the articles, devices and methods described herein can be practiced with embodiments other than those disclosed. The disclosed embodiments are presented for purposes of illustration and not limitation. One will also understand that components of the devices, systems and methods depicted and described with regard the figures and embodiments herein may be interchangeable.

1. A non-invasive method for determining whether an expandable medical device is appropriate for implanting in a conduit of patient, comprising:
imaging a portion of the conduit of the patient into which the medical device is to be implanted, wherein the imaging is performed during a plurality of portions of the cardiac cycle including (i) a portion of the cardiac cycle at which one or more dimensional characteristics of the conduit are at or near minimums and (ii) a portion of the cardiac cycle at which the one or more dimensional characteristics of the conduit are at or near maximums;
deriving, from the imaging, minimum and maximum dimensional characteristics of the conduit at a plurality of locations along a length of the portion of the conduit; and
determining that the medical device is appropriate for implantation if the maximum and minimum dimensional characteristics of the conduit at the predetermined locations along the length of the conduit are within predetermined ranges of maximum, expanded and minimum, contracted dimensional characteristics of the medical device at predetermined locations along the length of the medical device.

2. A method according to claim 1, wherein the medical device is a transcatheter medical device.

3. A method according to claim 1, wherein the medical device comprises a prosthetic heart valve device having a proximal inflow region and a distal outflow region.

4. A method according to claim 3, wherein the conduit comprises a heart valve.

5. A method according to claim 4, wherein imaging comprises capturing image data at or near 30% phase and at or near 90% phase of a cardiac cycle of the patient.

6. A method according to claim 4, wherein the medical device is determined to be appropriate for implantation if the maximum dimensional characteristic of the conduit at a predetermined location along the length of the conduit is 60% to 95% of the maximum dimensional characteristic of the medical device at a location along a distal outflow portion of the medical device having the largest dimensional characteristic of the distal outflow portion of the medical device.

7. A method according to claim 4, wherein the medical device is determined to be appropriate for implantation if the maximum dimensional characteristic of the conduit at a predetermined location along the length of the conduit is 85% or less than the maximum dimensional characteristic of the medical device at a location along a distal outflow portion of the medical device having the largest dimensional characteristic of the distal outflow portion of the medical device.

8. A method according to claim 4, wherein the medical device is determined to be appropriate for implantation if the maximum dimensional characteristic of the conduit at a predetermined location along the length of the conduit is 60% to 90% of the maximum dimensional characteristic of the medical device at a location along a proximal inflow portion of the medical device having the largest dimensional characteristic of the proximal inflow portion of the medical device.

9. A method according to claim 4, wherein the conduit is a pulmonary artery.

10. A method according to claim 1, wherein the imaging comprises magnetic resonance imaging, ultrasound imaging, computed tomography imaging, or x-ray imaging.

11. A method according to claim 1, wherein the method is a computer-implemented method.

12. A computer implemented method for determining whether an expandable medical device is appropriate for implanting in a conduit of patient, comprising:
receiving input of imaging data regarding a portion of the conduit of the patient into which the medical device is to be implanted, wherein the imaging data comprises data obtained during a plurality of portions of the cardiac cycle, including (i) a portion of the cardiac cycle at which dimensional characteristics of the conduit are at or near minimums and (ii) a portion of the cardiac cycle at which the dimensional characteristics of the conduit are at or near maximums;
calculating minimum and maximum dimensional characteristics of the conduit at a plurality of locations along a length of the portion of the conduit based on the imaging data; and
determining whether the maximum and minimum dimensional characteristics of the conduit at the predetermined locations along the length of the conduit are within predetermined ranges of maximum, expanded and minimum, contracted dimensional characteristics of the medical at predetermined locations along the length of the medical.

13. A method according to claim 12, wherein the medical device is a transcatheter medical device.

14. A method according to claim 12, wherein the medical device comprises a prosthetic heart valve.

15. A method according to claim 12, further comprising receiving input regarding the length and maximum, expanded and minimum, contracted dimensional characteristics of the medical device at predetermined locations along the length of the medical device.

16. A method according to claim 15, further comprising displaying data indicative of whether the medical device is appropriate for implanting in the conduit of the patient.

17. A non-transitory computer-readable medium that when executed by a device causes the device to carry out a method according to claim 12.

18. A device for determining whether an expandable medical device is appropriate for implanting in a conduit of patient, the device comprising:
input apparatus configured to receive input of imaging data regarding a portion of the conduit of the patient into which the medical device is to be implanted, wherein the imaging data comprises data obtained during a plurality of portions of the cardiac cycle, including (i) a portion of the cardiac cycle at which dimensional characteristics of the conduit are at or near minimums and (ii) a portion of the cardiac cycle at which the dimensional characteristics of the conduit are at or near maximums; and
computing apparatus configured to:
calculate minimum and maximum dimensional characteristics of the conduit at a plurality of locations along a length of the portion of the conduit based on the imaging data; and
determining whether the maximum and minimum dimensional characteristics of the conduit at the predetermined locations along the length of the conduit are within predetermined ranges of maximum, expanded and minimum, contracted dimensional characteristics of the medical device at predetermined locations along the length of the medical device.

19. A device according to claim 18, wherein the medical device is a transcatheter medical device comprising a prosthetic heart valve.

20. A device according to any one of claims 18, wherein the input apparatus is further configured to receive input regarding the length and maximum, expanded and minimum, contracted dimensional characteristics of the medical at predetermined locations along the length of the medical device.
